(19) United States
(12) Patent Application Publication
(10) Pub. No.: US 2009/0043169 A1
(43) Pub. Date: Feb. 12, 2009

(54) Childbirth instrument and method

(75) Inventors: Hai H. Trieu, Cordova, TN (US); David Lee Grant, Bartlett, TN (US); William L. Waltersdorff, Hernando, MS (US); Charles Tyson Dunn, Cordova, TN (US); Christopher Edward Johnson, Germantown, TN (US)

Correspondence Address: RUSSELL H. WALKER;WALKER, McKENZIE & WALKER, P.C., 6363 POPLAR AVE., Suite 318, MEMPHIS, TN, 38119-4899, US

(73) Assignee: IMS Launch LLC, Bartlett, TN (US)

(21) Appl. No.: 12/228,159

(22) Filed: Aug. 8, 2008

Related U.S. Application Data
(60) Provisional application No. 60/964,095, filed on Aug. 9, 2007.

(51) Int. Cl. A61B 1/32

(52) U.S. Cl. 600/220

(57) ABSTRACT

A childbirth instrument and method of use. The instrument may have guide and/or guard portions with concave posterior sides, and may have one or more grips. The guide may have first, second, and third elongated reference slots for performing episiotomy cuts therethrough, with the reference slots locating the angle and position of the episiotomy cuts, and the guard provides protection for the unborn infant. Another embodiment has one or more apertures through which a hypodermic needle may deliver a local anesthetic prior to making episiotomy cuts. The instrument may have one or more grips, which may be removable, and may have a movable stop and/or guide aperture. The angle of the episiotomy cut may be adjusted with one embodiment. The guide and guard portions may separate. Finger-receiving portions may be provided on the guard.

### CROSS REFERENCE TO RELATED APPLICATIONS

This application is a non-provisional continuation-in-part, and claims priority benefit, of U.S. Provisional Patent Application No. 60/964095 (filed Aug. 9, 2007) entitled “Innovative Medical Solutions Episiotomy Assistance Instrument”, hereby specifically incorporated herein by reference in its entirety.

### STATEMENT REGARDING FEDERALLY SPONSORED RESEARCH OR DEVELOPMENT

Not applicable.

### REFERENCE TO COMPACT DISC(S)

Not applicable.

### BACKGROUND OF THE INVENTION

1. Field of the Invention

The present invention relates, in general, to medical instrumentation used in childbirth, and in particular, to medical instrumentation for use in childbirth and methods of use of same.

2. Information Disclosure Statement

Episiotomies represent a common surgical procedure performed each year in the United States and abroad Episiotomies are typically an unplanned surgical procedure performed during vaginal delivery when the attending physician/midwife believes that either the mother or unborn child is at risk and also believes the episiotomy will speed up the delivery. Additionally, it is thought that performing an episiotomy will help prevent anterior perinea lacerations.

Heretofore, attending physicians and midwives have had only two instrument options when an episiotomy is required, namely, surgical scissors or scalpels. While no clinical studies have been conducted to determine which of these instruments is most efficient, the literature suggests that scissors are the primary instrument of choice.

There are inherent problems or shortcomings with prior art episiotomy scissors. While they can cut easily through the perineum, they offer no means to accurately gauge the length and angle of the episiotomy cut. Persons performing the procedure can only make an educated guess as to the length and angle of the episiotomy incision. The published literature suggests these two guesses represent two of the most critical aspects as to whether the episiotomy procedure will be successful.

Patient anatomy varies from woman to woman, with the perineum ranging in length from about 2.5 cm to about 7 cm. Research has suggested that the length of the perineum, coupled with the angle of the episiotomy, are factors in determining the risk of adverse events, both for mother and unborn child, associated with episiotomy. Adverse events to the mother commonly associated with episiotomy include third and fourth degree lacerations, endometriosis at the episiotomy site, granular cell tumor of the vulva in the episiotomy scar, increased blood loss, hematoma pain, and edema. These adverse events often require follow-up medical care and can lead to long-term afflictions such as anal incontinence. Adverse events to the unborn child commonly associated with episiotomy include eyelid laceration and, on some occasions, when the birth is breech, castration.

Heretofore, when applying a local anesthetic to the perineum prior to performing the episiotomy procedure, an attending physician and/or midwife would insert his/her gloved index and second fingers into the woman's vagina between the unborn child's head and the inner surface of the vagina, and spread the vaginal entrance while lifting the perineal surface structure away from the unborn child's head during injection of a local anesthetic such as lidocaine into the perineum. This prior art method of anesthetizing the perineum can permit accidental injury to the unborn infant's unprotected head, and, because the attending physician's or midwife's fingers are beneath the perineal surface being injected with anesthesia, may allow those fingers to be accidentally pricked or injected by the anesthesia syringe's needle.

It is therefore desirable to provide a childbirth instrument and method of use that enables an attending physician and/or midwife to perform an episiotomy procedure with greater accuracy in length and placement of episiotomy cuts, and which reduces the occurrence of injury to an unborn child during the episiotomy procedure.

It is further desirable to provide a childbirth instrument and method of use that reduces the risk of injury to an unborn child and to an attending physician or midwife during injection of anesthesia into the perineum prior to performing an episiotomy procedure or other medical procedures in the vaginal area.

Agmon et al., U.S. Patent Application Publication No. 2008/0097472A1 (published Apr. 24, 2008), describes a flexible sheet for use in performing an episiotomy, and assists the attending physician in locating the episiotomy incision.

Bacon, U.S. Pat. No. 1,894,725 (issued Jan. 17, 1933), discloses a speculum having a pivoted pair of opposed jaws with handles.

Davis, U.S. Pat. No. 3,796,214 (issued Mar. 12, 1974), discloses a perineal retractor for insertion into the vagina.

Salas-Ceniceros, U.S. Pat. No. 5,139,503 (issued Aug. 18, 1992), discloses an obstetrical spatula used to assist vaginal exit of an infant during childbirth.

Auerbach et al., U.S. Pat. No. 6,302,842 (issued Oct. 16, 2001), discloses an episiotomy retractor having a pivoted pair of opposed blades with handles for retraction of vaginal walls.

No prior art references, either singly or in combination, are believed to disclose or suggest the present invention.

### BRIEF SUMMARY OF THE INVENTION

Various embodiments of the childbirth instrument of the present invention are provided, and all embodiments are sized and adapted for use in the region of the vagina. Many embodiments have a guide portion with a reference slot with adjacent reference edge for assisting the attending physician or midwife in placement of the episiotomy incision and in determining the proper length for the episiotomy cut, and some embodiments may have a plurality of guide reference slots or guide reference edges for locating the episiotomy incision placement. The instrument may also have three guide reference slots so that two opposing episiotomy incisions may be made without repositioning of the childbirth instrument or so that alternately, and more commonly, a physician or midwife could choose whether to make a single episiotomy incision to the left or to the right of the anus after a single positioning of the childbirth instrument. Other embodiments have a guard portion that is inserted into the vagina for protecting the unborn child, and an aperture may be provided adjacent an upper portion of the guard to permit perineal access to a hypodermic needle for delivery of a local anesthetic to the perineum. Some embodiments have both a guide portion and a guard portion spaced apart from the guide portion, and a handle or grip may be optionally provided to ease the placement and use of the instrument during the episiotomy procedure. Reference markings may be provided with some embodiments to assist in determining the length and angle of the episiotomy incision, and a stop may be provided with the instrument to limit the length and/or depth of the episiotomy incision. The instrument may be affixed adjacent the episiotomy incision site by docking the instrument onto the vagina, anus and/or surrounding skin surfaces on the perineum, or through the use of a mild adhesive such as a glue or on an adhesive backing or tape, to bind the instrument to the surface of the perineum. Alternatively, if desired, well-known hooks, plugs, straps or tethers may be used to affix the instrument to the perineum adjacent the vagina.

The childbirth instrument of the present invention is preferably a single-use, sterile instrument that is discarded after the episiotomy procedure has concluded. The instrument may be made either from plastics and/or metal and may be provided in various sizes adapted to fit the anatomy of a patient.

A method of using the childbirth instrument is also provided, whereby the instrument is placed at the episiotomy incision site and is then used in the episiotomy procedure to assist the attending physician or midwife in making the episiotomy incision safely and accurately, reducing the occurrence of injury to the mother and to the unborn child.

It is an object of the present invention to provide a childbirth instrument and method of use that enables an attending physician and/or midwife to perform an episiotomy procedure with greater accuracy in length and placement of episiotomy cuts than heretofore possible in the prior art, and which reduces the occurrence of injury to an unborn child during the episiotomy procedure.

It is a further object of the present invention to provide a childbirth instrument and method of use that reduces the risk of injury to an unborn child and to an attending physician or midwife during injection of anesthesia into the perineum prior to performing an episiotomy procedure. It is an object of some embodiments of the present invention to provide a guard to reduce the risk of injury to the unborn infant from the scalpel or surgical scissors used to make the episiotomy incisions, and/or from the hypodermic needle used for delivering local anesthesia to the perineum.

It is a further object of the present invention to provide the attending physician and/or midwife with an instrument to quickly measure the perineum and to set a precise angle and length for the episiotomy incisions.

### BRIEF DESCRIPTION OF THE SEVERAL VIEWS OF THE DRAWING

FIG. 1 is an anterior view of a first embodiment of the childbirth instrument of the present invention.

FIG. 2 is a lateral view of the first embodiment of the childbirth instrument of the present invention.

FIG. 3 is a posterior view of the first embodiment of the childbirth instrument of the present invention.

FIG. 4 is a perspective view of the first embodiment of the childbirth instrument of the present invention.

FIG. 4A is an anterior view of a variation of the first embodiment of the childbirth instrument of the present invention showing optional reference dimensional and angular markings.

FIG. 5 is an anterior view of a second embodiment of the childbirth instrument of the present invention.

FIG. 6 is a posterior view of the second embodiment of the childbirth instrument of the present invention.

FIG. 7 is an anterior view of a third embodiment of the childbirth instrument of the present invention.

FIG. 8 is a lateral view of the third embodiment of the childbirth instrument of the present invention.

FIG. 9 is a posterior view of the third embodiment of the childbirth instrument of the present invention.

FIG. 10 is a perspective view of the third embodiment of the childbirth instrument of the present invention.

FIG. 11 is an anterior view of a fourth embodiment of the childbirth instrument of the present invention.

FIG. 12 is a lateral view of the fourth embodiment of the childbirth instrument of the present invention.

FIG. 13 is a posterior view of the fourth embodiment of the childbirth instrument of the present invention.

FIG. 14 is a perspective view of the fourth embodiment of the childbirth instrument of the present invention.

FIG. 15 is an anterior view of a fifth embodiment of the childbirth instrument of the present invention, with an attending physician's gloved fingers shown in dotted outline.

FIG. 16 is a lateral view of the fifth embodiment of the childbirth instrument of the present invention.

FIG. 17 is a posterior view of the fifth embodiment of the childbirth instrument of the present invention, with an attending physician's gloved fingers shown in dotted outline.

FIG. 18 is a perspective view of the fifth embodiment of the childbirth instrument of the present invention.

FIG. 19 is an anterior view of a sixth embodiment of the childbirth instrument of the present invention.

FIG. 20 is a lateral view of the sixth embodiment of the childbirth instrument of the present invention.

FIG. 21 is a posterior view of the sixth embodiment of the childbirth instrument of the present invention, with an attending physician's gloved fingers shown in dotted outline.

FIG. 22 is a perspective view of the sixth embodiment of the childbirth instrument of the present invention.

FIG. 23 is an anterior view of a seventh embodiment of the childbirth instrument of the present invention.

FIG. 24 is a lateral view of the seventh embodiment of the childbirth instrument of the present invention, with an attending physician's gloved thumb shown in dotted outline.

FIG. 25 is a posterior view of the seventh embodiment of the childbirth instrument of the present invention.

FIG. 26 is a perspective view of the seventh embodiment of the childbirth instrument of the present invention.

FIG. 27 is a side sectional view of the seventh embodiment of the childbirth instrument of the present invention, taken substantially along the line 27-27 shown in FIG. 23.

FIG. 28 is an anterior view of an eighth embodiment of the childbirth instrument of the present invention.

FIG. 29 is a lateral view of the eighth embodiment of the childbirth instrument of the present invention, with an attending physician's gloved thumb shown in dotted outline.

FIG. 30 is a posterior view of the eighth embodiment of the childbirth instrument of the present invention.

FIG. 31 is a side sectional view of the eighth embodiment of the childbirth instrument of the present invention, taken substantially along the line 31-31 shown in FIG. 28.

FIG. 32 is a perspective view of the eighth embodiment of the childbirth instrument of the present invention.

FIG. 33 is an anterior view of a ninth embodiment of the childbirth instrument of the present invention.

FIG. 34 is an anterior view of a tenth embodiment of the childbirth instrument of the present invention.

FIG. 35 is an anterior view of an eleventh embodiment of the childbirth instrument of the present invention.

FIG. 36 is a lateral view of the eleventh embodiment of the childbirth instrument of the present invention.

FIG. 37 is a posterior view of the eleventh embodiment of the childbirth instrument of the present invention.

FIG. 38 is a perspective view of the eleventh embodiment of the childbirth instrument of the present invention.

FIG. 39 is an anterior view of a twelfth embodiment of the childbirth instrument of the present invention.

FIG. 40 is a lateral view of the twelfth embodiment of the childbirth instrument of the present invention.

FIG. 41 is a posterior view of the twelfth embodiment of the childbirth instrument of the present invention.

FIG. 42 is a perspective view of the twelfth embodiment of the childbirth instrument of the present invention.

FIG. 43 is an anterior view of a thirteenth embodiment of the childbirth instrument of the present invention.

FIG. 44 is a lateral view of the thirteenth embodiment of the childbirth instrument of the present invention.

FIG. 45 is a posterior view of the thirteenth embodiment of the childbirth instrument of the present invention.

FIG. 46 is a perspective view of the thirteenth embodiment of the childbirth instrument of the present invention.

FIG. 47 is a perspective view of a fourteenth embodiment of the childbirth instrument of the present invention.

FIG. 48 is an anterior view of a fifteenth embodiment of the childbirth instrument of the present invention.

FIG. 49 is a lateral view of the fifteenth embodiment of the childbirth instrument of the present invention.

FIG. 50 is a perspective view of the fifteenth embodiment of the childbirth instrument of the present invention.

FIG. 51 is a posterior view of the fifteenth embodiment of the childbirth instrument of the present invention.

FIG. 52 is a perspective view of a sixteenth embodiment of the childbirth instrument of the present invention.

FIG. 53 is a perspective view of a seventeenth embodiment of the childbirth instrument of the present invention showing separation of the removable grip.

FIG. 54 is another perspective view of the seventeenth embodiment of the childbirth instrument of the present invention showing attachment of the removable grip.

FIG. 55 is a perspective view of an eighteenth embodiment of the childbirth instrument of the present invention.

FIG. 56 is a lateral view of the eighteenth embodiment of the childbirth instrument of the present invention.

FIG. 57 is an anterior view of a nineteenth embodiment of the childbirth instrument of the present invention.

FIG. 58 is a lateral view of the nineteenth embodiment of the childbirth instrument of the present invention.

FIG. 59 is a perspective view of the nineteenth embodiment of the childbirth instrument of the present invention.

FIG. 60 is a posterior view of the nineteenth embodiment of the childbirth instrument of the present invention.

FIG. 61 is a perspective view of a twentieth embodiment of the childbirth instrument of the present invention.

FIG. 62 is an anterior view of a twenty-first embodiment of the childbirth instrument of the present invention.

FIG. 63 is a lateral view of the twenty-first embodiment of the childbirth instrument of the present invention.

FIG. 64 is a perspective view of the twenty-first embodiment of the childbirth instrument of the present invention.

FIG. 65 is another perspective view of the twenty-first embodiment of the childbirth instrument of the present invention but with the parts shown separated.

FIG. 66 is a side sectional view of the twenty-first embodiment of the childbirth instrument of the present invention, taken substantially along the line 66-66 shown in FIG. 64.

FIG. 67 is an anterior view of a twenty-second embodiment of the childbirth instrument of the present invention, showing, in dotted outline, movement of the guide's reference arm.

FIG. 68 is a lateral view of the twenty-second embodiment of the childbirth instrument of the present invention.

FIG. 69 is a perspective view of the twenty-second embodiment of the childbirth instrument of the present invention.

FIG. 70 is an anterior view of a twenty-third embodiment of the childbirth instrument of the present invention, showing, in dotted outline, movement of the guide stop.

FIG. 71 is a lateral view of the twenty-third embodiment of the childbirth instrument of the present invention.

FIG. 72 is a perspective view of the twenty-third embodiment of the childbirth instrument of the present invention.

FIG. 73 is a sectional view of the twenty-third embodiment of the childbirth instrument of the present invention, taken substantially along the line 73-73 shown in FIG. 72.

FIG. 74 is a perspective view of a twenty-fourth embodiment of the childbirth instrument of the present invention, showing, in dotted outline, movement of the guide member.

FIGS. 75 and 76 are perspective views of the twenty-fourth embodiment of the childbirth instrument of the present invention, showing insertion of a scalpel in the guide aperture of the movable guide member.

FIG. 77 is a perspective view of a twenty-fifth embodiment of the childbirth instrument of the present invention.

FIG. 78 is an anterior view of a twenty-sixth embodiment of the childbirth instrument of the present invention.

FIG. 79 is a lateral perspective view of a twenty-seventh embodiment of the childbirth instrument of the present invention.

FIG. 80 is an anterior view of the twenty-seventh embodiment of the childbirth instrument of the present invention.

FIG. 81 is a view showing placement of the first embodiment of the childbirth instrument in a vagina in a first position and, in dotted outline, showing placement of the childbirth instrument in the vagina in a second position.

FIG. 82 is a view showing placement of the twenty-sixth embodiment of the childbirth instrument in a vagina in a first position and, in dotted outline, showing placement of the childbirth instrument in the vagina in a second position.

FIG. 83 is a view showing placement of the second embodiment of the childbirth instrument in a vagina.

FIG. 84 is a side sectional view through a vagina showing use of the childbirth instrument of the present invention.

FIG. 85 shows an alternate version of the view of FIG. 81, and is a view showing placement of the first embodiment of the childbirth instrument in a vagina in an alternate first position and, in dotted outline, showing placement of the childbirth instrument in the vagina in an alternate second position.

### DETAILED DESCRIPTION OF THE INVENTION

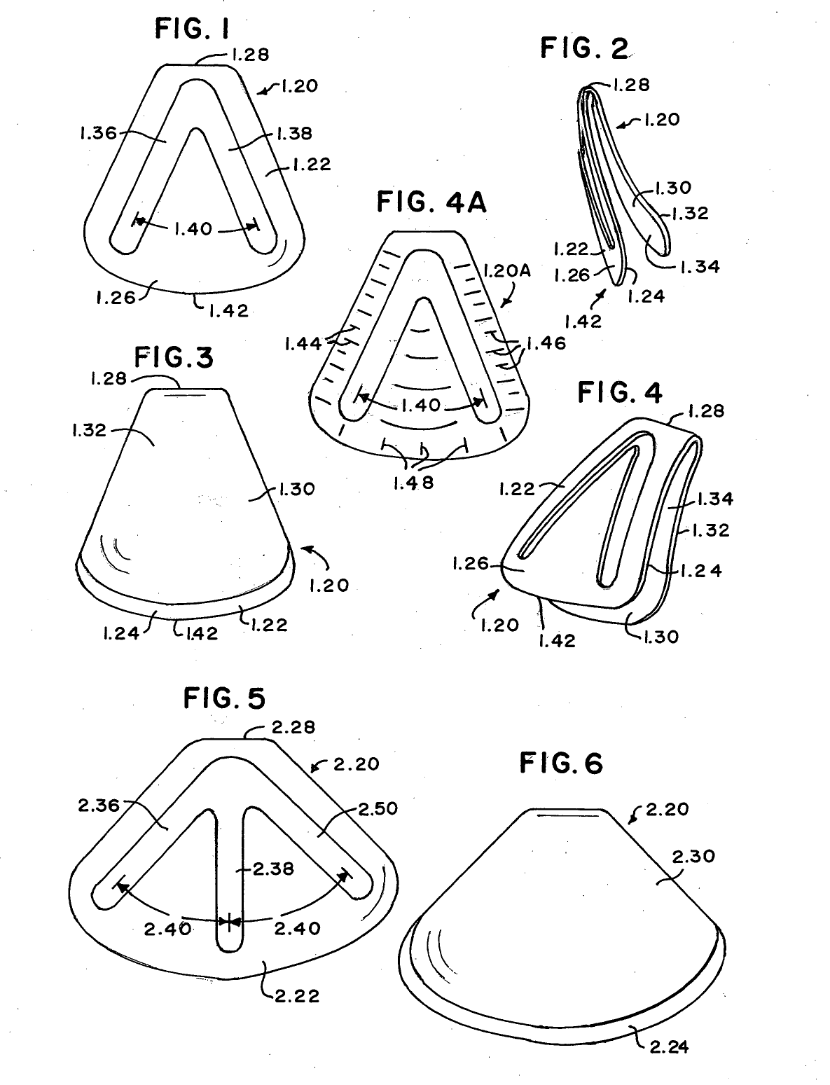

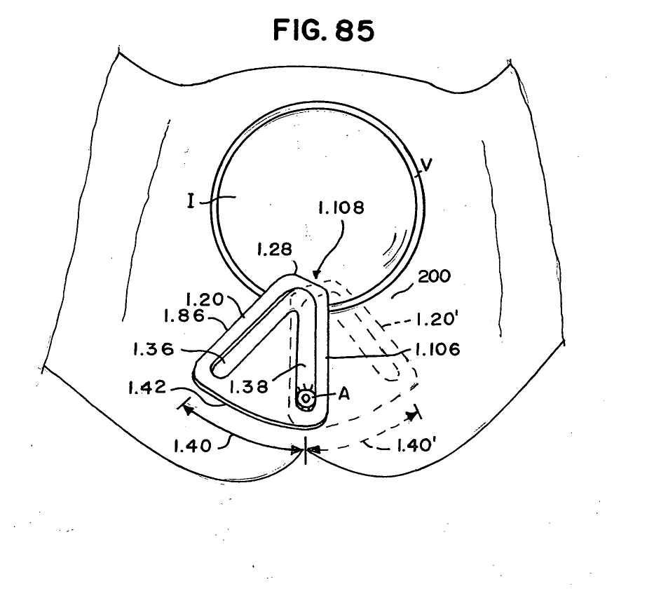

Referring to FIGS. 1-85, many preferred embodiments of the childbirth instrument of the present invention are shown. Identifying reference designators for all embodiments of the childbirth instrument are marked similarly, with the reference designators for the various embodiments respectively having prefixes of “1.”, “2.”, “3.”, etc., and with similar structural features of the various embodiments having the same suffix (e.g., “1.20”, “2.20”, “3.20”, etc.). It shall be understood that many aspects of the various preferred embodiments are substantially the same, and only the differences will be treated in detail, it being understood that similar structural features of the various embodiments perform similar functions.

Each embodiment of the present invention is preferably a single-use, sterile instrument that is discarded once the episiotomy procedure has concluded. All embodiments are constructed of well-known materials suitable for sterilization, and are sized and adapted to fit the perineal and vaginal anatomy of a woman patient.

All embodiments preferably have rounded edges, corners, etc., and are devoid of sharp and/or pointed features which may pose a risk of injury to the unborn child or cause injury or discomfort to the mother. All embodiments may be rigid, semi-rigid and/or flexible for optimal performance and comfort to the patient and unborn child.

All embodiments may have surface coatings or laminates for various purposes including increased comfort, enhanced lubricity, fluid absorption capability, anti-fouling feature, anti-bacterial function, etc. As is well-known to those skilled in the art, the surfaces of all embodiments are preferably compatible with, or can accommodate, application of pharmaceutical agents such as antibiotics, analgesics, anesthetics, etc.

All embodiments may be clear, translucent and/or opaque and may be either a solid color or a combination of colors, and all embodiments may be made of one or more materials including plastics, elastomers, rubbers, metals, ceramics and/or composites. Examples of suitable plastic materials include polycarbonates, acrylics, polyvinyl chloride, polystyrene, polyethylene, poly propylene, nylon, ABS, etc. Examples of suitable elastomers include well-known silicone, polyurethane, polyolefin, etc.

All embodiments may be manufactured by one or more methods which include injection molding, compression molding, reactive injection molding, thermal forming, machining, etc. Manufacturing of all embodiments also includes various assembly steps, packaging and sterilization. Sterilization methods can be gamma irradiation, ethylene oxide gas, gas plasma, autoclaving and chemical disinfectants, etc., all well-known to those skilled in the art.

Suitable material for use in fabricating all embodiments include well-known plastics, elastomers, rubbers, metals, ceramics and/or composites. Examples of suitable well-known plastic materials include polycarbonates, acrylics. All embodiments may be provided in a range of sizes as required to adapt to the anatomy of a specific patient.

Referring to FIGS. 1-4, a first embodiment 1.20 of the childbirth instrument of the present invention is shown.

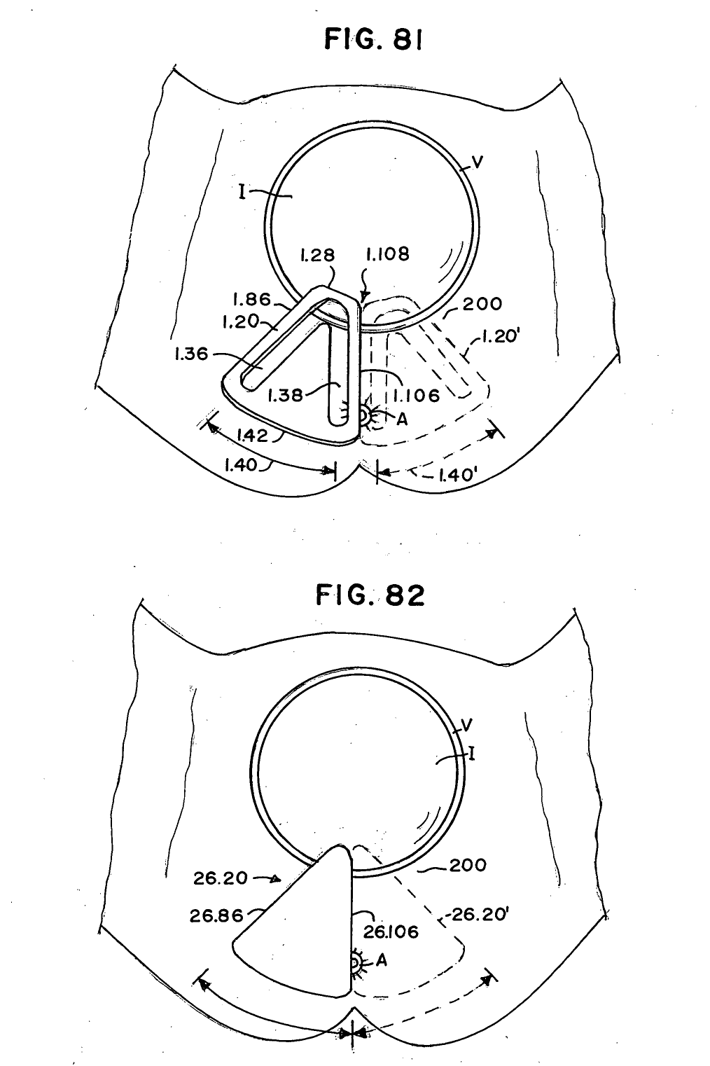

First embodiment 1.20 includes a guide portion 1.22, preferably substantially thin so as to permit close access to the perineum by the attending physician and/or midwife, and adapted for fitting against a woman's perineal outer surface 200 (see FIGS. 81 and 84) with guide portion 1.22 having a concave posterior side 1.24 and a preferably convex anterior side 1.26. The top or vaginal end 1.28 of the instrument 1.20 is preferably narrowed to permit retention in the vagina when the childbirth instrument is docked therewithin as shown, for example, in FIGS. 81 and 84, described in more detail hereinafter.

First embodiment 1.20 also includes a substantially thin guard portion 1.30 adapted for insertion into a woman's vagina, with guard portion 1.30 having a concave posterior side 1.32 for being received adjacent the unborn infant and a convex anterior side 1.34 for being received against the inner wall of the vagina, with guard portion 1.30 being intermediate the unborn infant and the inner wall of the vagina for protection of the unborn infant.

Guide portion 1.22 has an elongated first reference slot 1.36 therethrough and also has an elongated second reference slot 1.38 therethrough, with first and second reference slots 1.36, 1.38 being at a selected first angle 1.40 with respect to each other and first and second reference slots 1.36, 1.38 being in communication with each other proximate the top or vaginal end 1.28 of instrument 1.20. While the reference slots are shown as being straight in the preferred embodiments, it shall be understood that the reference slots may be slightly curved or aggressively curved into a J-shape (not shown) in order to guide the creation of episiotomy incisions without departing from the spirit and scope of the present invention. The width of the reference slots is chosen and sized to allow for ease of insertion and operation of the episiotomy cutting instrument, be it scissors or scalpel, at the episiotomy incision site. First angle 1.40 may be any angle between five degrees and seventy-five degrees, inclusive, but the preferred angle is between fifteen and sixty degrees, inclusive, and the optimal angle is between thirty and forty-five degrees, inclusive.

Guard portion 1.30 is joined to guide portion 1.22 at the vaginal end 1.28 of instrument 1.20 and guard and guide portions 1.30, 1.22 are spaced apart from each other, with the convex anterior side 1.34 of guard portion 1.30 being opposed from posterior side 1.24 of guide portion 1.22. As hereinafter described in detail, when docked with a vagina, the bottom or anus end 1.42 of instrument 1.20 will be proximate a woman's anus A as seen in FIGS. 81 and 84, such that the reference slots converge toward the vagina and diverge toward the anus. It shall be understood that, with all embodiments having both a guide portion and a guard portion, the guard portion, which may be relatively longer or shorter than shown in the drawings, preferably extends below the reference slots of the guide portion so as to provide protection for the unborn infant when surgical instruments are inserted through the reference slots in the guide portion while performing the episiotomy procedure, as hereinafter described in greater detail. With all embodiments having a guard portion, the guard portion is designed to function as a barrier to prevent, or reduce the possibility of, injury to the unborn infant in the event the cutting instrument happens to extend further toward the unborn infant than intended. The guard portion is designed to be large enough to cover the area of the unborn infant's body that might be exposed to the cutting instrument. If desired, the guard may be provided with a soft plastic or rubberized hook, shoulder or stopper to prevent migration and excessive movement, and to help retrieve or remove the instrument following completion of the episiotomy procedure.

FIG. 4A shows a variation 1.20A of the first embodiment 1.20 in which optional reference linear dimensional markings 1.44 and 1.46, in metric or English measurement units, may be provided adjacent one or both of the elongated first and second reference slots 1.36, 1.38. Also shown are optional reference angular markings 1.48 in degrees or other units within first angle 1.40. These reference markings provide a precise and easily-identifiable guide for the attending physician/midwife when making the episiotomy cuts, and may be raised to provide tactile feedback.

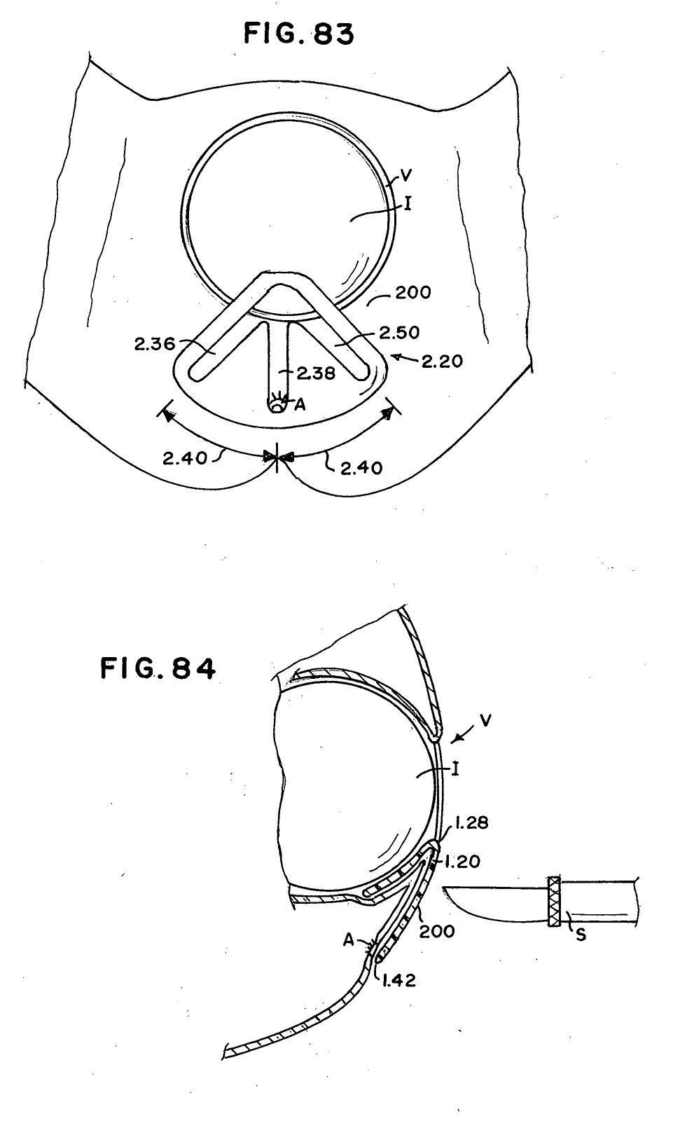

Referring to FIGS. 5 and 6, a second embodiment 2.20 of the childbirth instrument of the present invention is shown. Second embodiment 2.20 is similar to first embodiment 1.20, having a guard portion 2.30 and a guide portion 2.22, except that second embodiment 2.20 has elongated first, second, and third reference slots 2.36, 2.38, and 2.50 through guide portion 2.22, with second reference slot 2.38 being intermediate first and third reference slots 2.36, 2.50, and with first, second, and third reference slots 2.36, 2.38, and 2.50 being in mutual communication proximate the top/vaginal end 2.28 of second embodiment 2.20. The acute angle 2.40 between first and second reference slots 2.36, 2.38 is preferably the same as the acute angle 2.40 between second and third reference slots 2.38, 2.50, such that, with second reference slot 2.38 being placed in alignment with the woman's anus, two episiotomy cuts may be made through first and third reference slots 2.36 and 2.50 respectively without repositioning the childbirth instrument 2.20, as shown in FIG. 83 and as described in greater detail hereinafter. Alternatively, the second reference slot 2.38 may be used, if desired, to perform a mid-line episiotomy incision. More commonly, though, only a single episiotomy cut is made, rather than two episiotomy cuts, and this single episiotomy cut is typically made either to the left or the right of the anus, at the option of the attending physician/midwife, at his/her option. The second embodiment 2.20 of the childbirth instrument of the present invention permits the instrument 2.20 to be positioned once, with the second reference slot 2.38 in alignment with the woman's anus, and then a chosen one of first and third reference slots 2.36 and 2.50 is used to make the episiotomy incision, at the option of the attending physician/midwife. Some attending physicians/midwives may prefer the use of two shorter episiotomy incisions rather than a single longer episiotomy incision, and the second embodiment 2.20 of the childbirth instrument of the present invention accommodates such a procedure creating two episiotomy incisions without repositioning of the instrument 2.20.

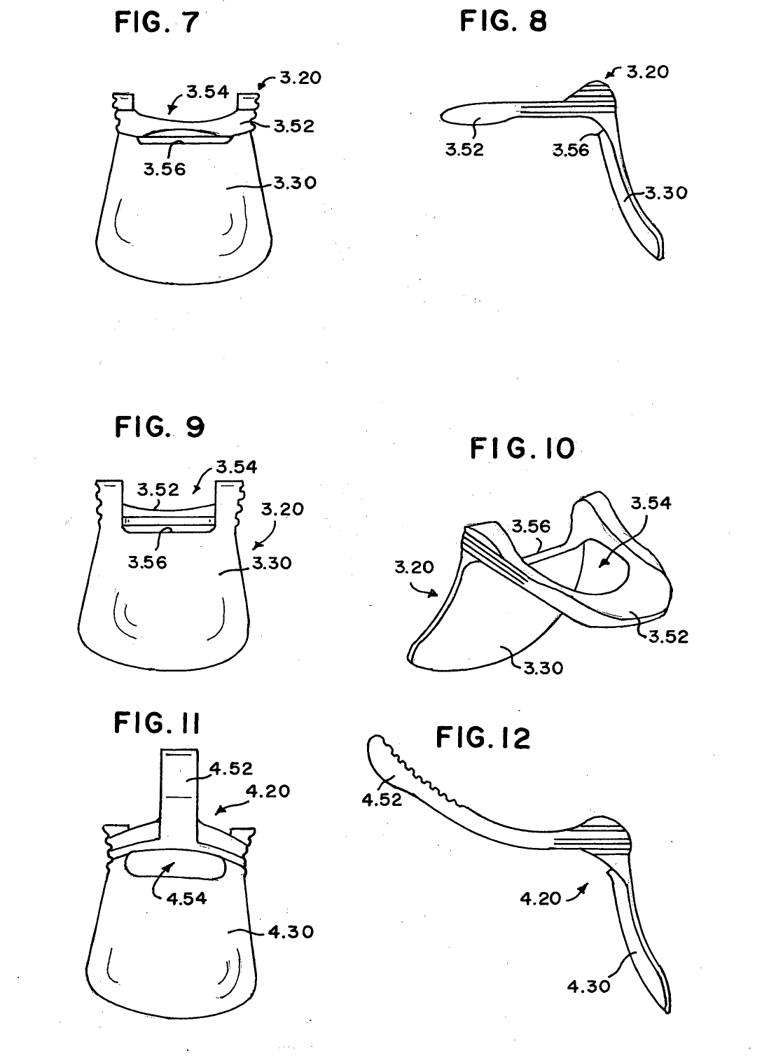

Referring to FIGS. 7-10, a third embodiment 3.20 of the childbirth instrument of the present invention is shown. Third embodiment 3.20 has a guard portion 3.30 similar to the guard portion of the first embodiment but has no guide portion, and has a handle or grip 3.52 that extends remotely from an upper portion 3.56 of the guard portion 3.30 for gripping by the physician to hold the instrument 3.20 in position. As before, the guard portion 3.30 is placed in the vagina intermediate the infant and the inner wall of the vagina. An aperture 3.54 is provided through instrument 3.20 proximate an upper portion 3.56 of guard portion 3.30 and proximate grip 3.52 to allow delivery of localized anesthesia through a hypodermic needle inserted through aperture 3.54 into the perineum.

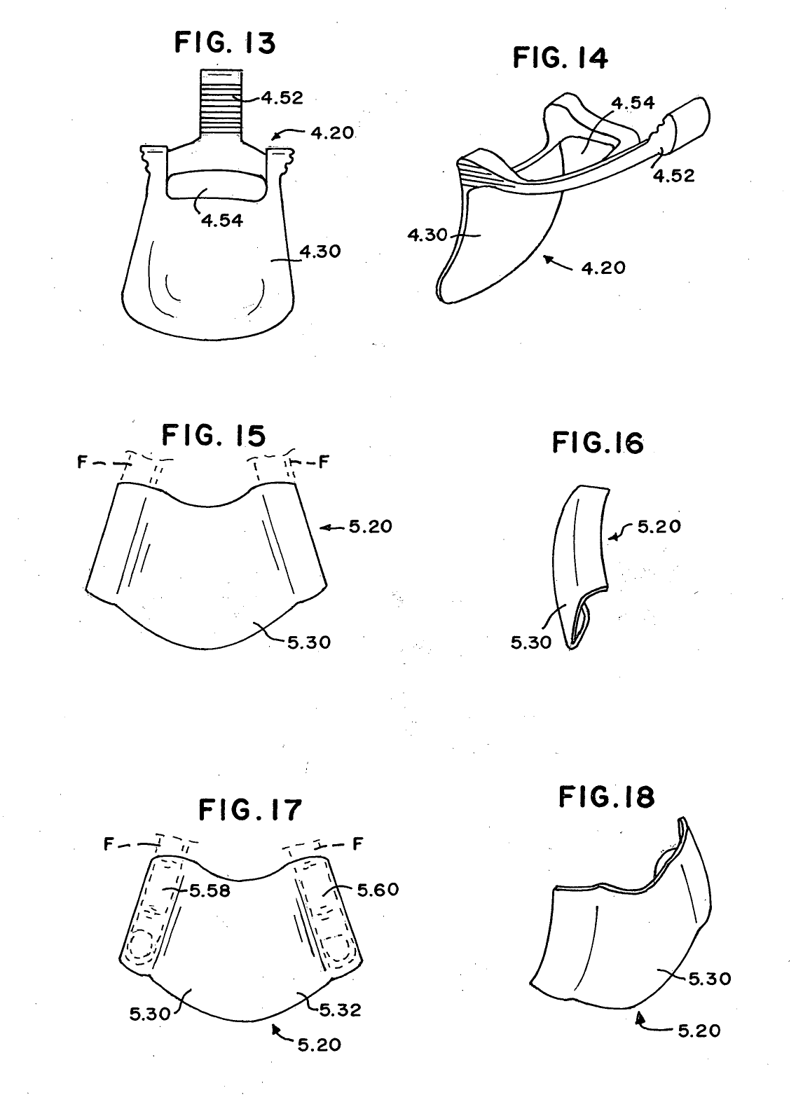

Referring to FIGS. 11-14, a fourth embodiment 4.20 of the childbirth instrument of the present invention is shown. Fourth embodiment 4.20 is similar to the third embodiment, the difference being in the structure of grip 4.52 as compared to grip 3.52. Whereas grip 3.52 has a low profile, grip 4.52 is larger to improve control in placement of the guard portion 4.30 in the vagina. Like the third embodiment, fourth embodiment 4.20 has an aperture 4.54 for delivery of localized anesthesia through a hypodermic needle into the perineum.

Referring to FIGS. 15-18, a fifth embodiment 5.20 of the childbirth instrument of the present invention is shown. Fifth embodiment 5.20 has a substantially thin guard portion 5.30, and guard portion 5.30 has a concave posterior side 5.32 and a convex anterior side 5.34. The guard portion 5.30 of instrument 5.20 also has spaced-apart first and second finger-receiving concave portions 5.58, 5.60 on the posterior side 5.32 of guard portion 5.30 for receiving a physician's index and second fingers F, with the physician's fingers being intermediate the unborn infant and guard portion 5.30 inside the vagina and urging the guard portion 5.30 against the inner wall of the vagina.

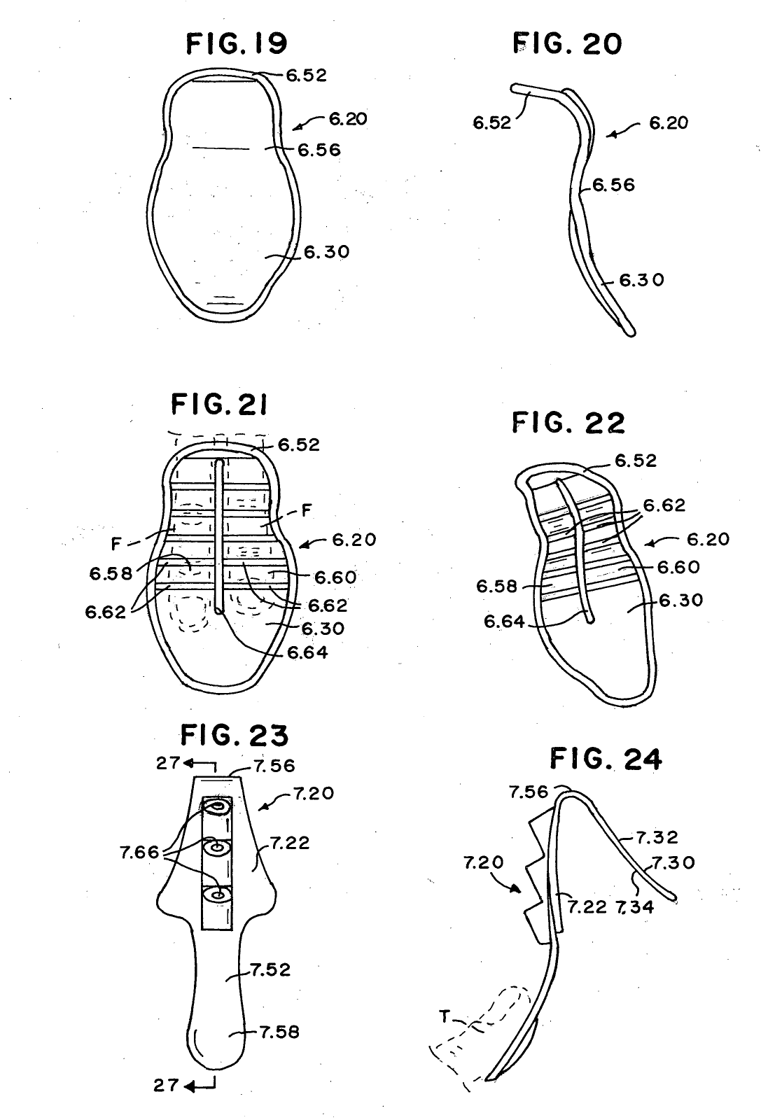

Referring to FIGS. 19-22, a sixth embodiment 6.20 of the childbirth instrument of the present invention is shown. Sixth embodiment 6.20 is similar to the fifth embodiment 5.20, but has a grip 6.52 that extends remotely from the upper portion 6.56 of the guard portion 6.30 for gripping by the physician to hold the instrument 6.20 in position. As with the fifth embodiment, sixth embodiment 6.20 has spaced-apart first and second finger-receiving concave portions 6.58, 6.60 on the posterior side 6.32 of guard portion 6.30 for receiving a physician's index and second fingers F, with the physician's fingers being intermediate the unborn infant and guard portion 6.30 inside the vagina and urging the guard portion 6.30 against the inner wall of the vagina. First and second finger-receiving portions 6.58, 6.60 also preferably have a plurality of transverse gripping bumps or ribs 6.62 for increased gripping by the physician's fingers, and a longitudinal rib 6.64 may be provided separating first and second finger-receiving portions 6.58, 6.60. It should be understood that the first and second finger-receiving portions 5.58, 5.60 may also have transverse ribs if desired without departing from the nature and scope of the present invention.

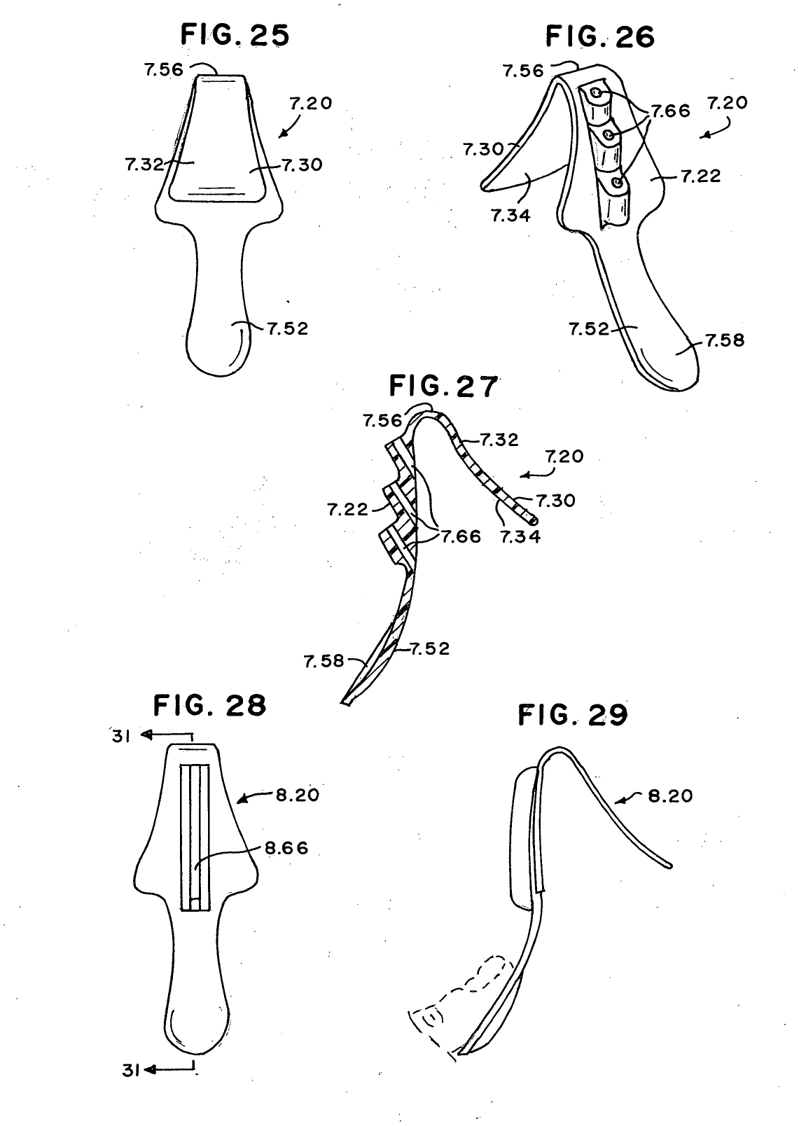

Referring to FIGS. 23-27, a seventh embodiment 7.20 of the childbirth instrument of the present invention is shown. The seventh embodiment 7.20 has a guide portion 7.22 adapted for fitting against a woman's perineal outer surface and a guard portion 7.30 adapted for insertion into a woman's vagina. Guard portion 7.30 is joined to guide portion 7.22 at an upper portion 7.56 and is spaced apart from guide portion 7.22. Guard portion 7.30 is substantially thin and has a concave posterior side, 7.32, and guard portion 7.30 has a convex anterior side 7.34 opposed from the posterior side 7.24 of guide portion 7.22. Guide portion 7.30 has at least a first aperture, and preferably a plurality of apertures, 7.66 therethrough adapted for receipt of a hypodermic needle thereinthrough to permit delivery of a local anesthetic to a woman's perineum when the hypodermic needle is received into one of the apertures 7.66 and into the woman's perineum. Seventh embodiment 7.20 also has a grip portion 7.52 having a finger-receiving concave portion 7.58 for receiving a physician's thumb T thereon, permitting the physician to press the instrument 7.20 against the woman's perineum as the local anesthesia is being administered.

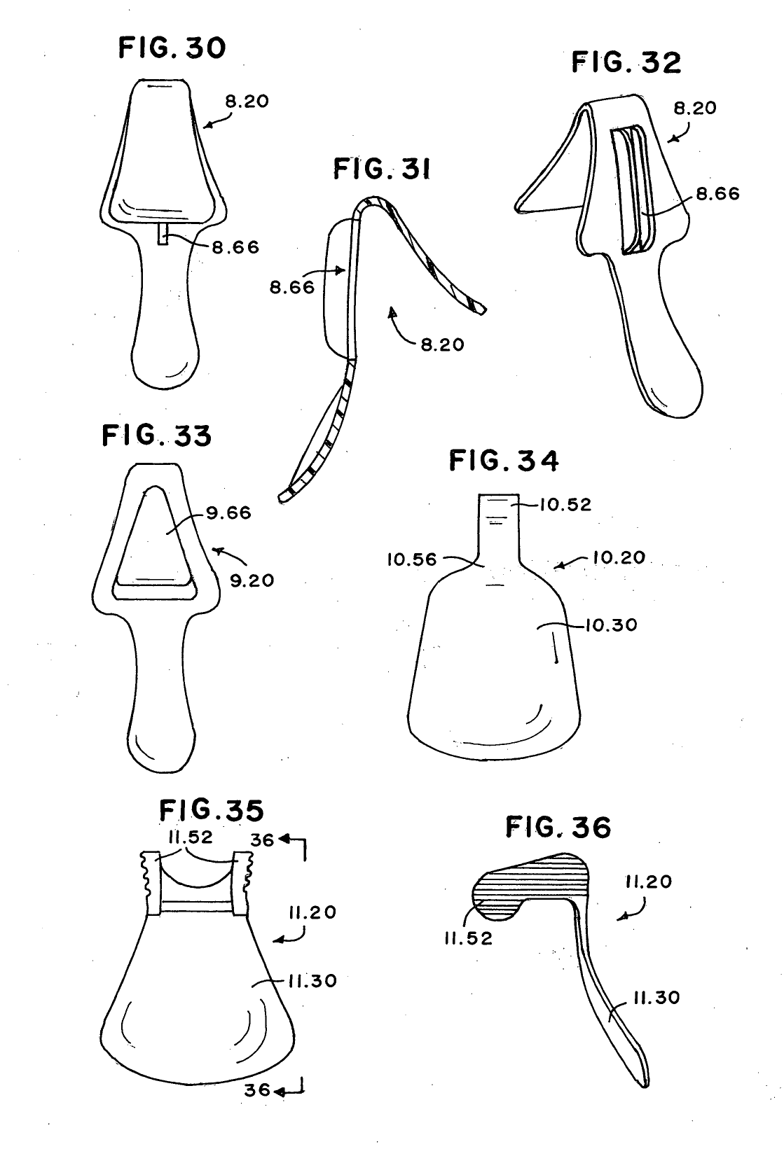

Referring to FIGS. 28-32, an eighth embodiment 8.20 of the childbirth instrument of the present invention is shown. Eighth embodiment 8.20 is substantially similar to seventh embodiment 7.20 except that eighth embodiment 8.20 has a single elongated slotted aperture 8.66 for receiving a hypodermic needle to administer local anesthesia rather than a plurality of cylindrical apertures 7.66 as in the seventh embodiment.

Referring to FIG. 33, a ninth embodiment 9.20 of the childbirth instrument of the present invention is shown. Embodiment 9.20 is substantially similar to seventh and eighth embodiments 7.20, 8.20 except that embodiment 9.20 has a single enlarged triangular aperture 9.66 for receiving a hypodermic needle therethrough to administer local anesthesia to the perineum.

Referring to FIG. 34, a tenth embodiment 10.20 of the childbirth instrument of the present invention is shown. Tenth embodiment 10.20 is substantially similar to fourth embodiment 4.20 except that tenth embodiment 10.20 has no aperture corresponding to aperture 4.54, and grip 10.52 is centrally joined to upper portion 10.56 of guard portion 10.30.

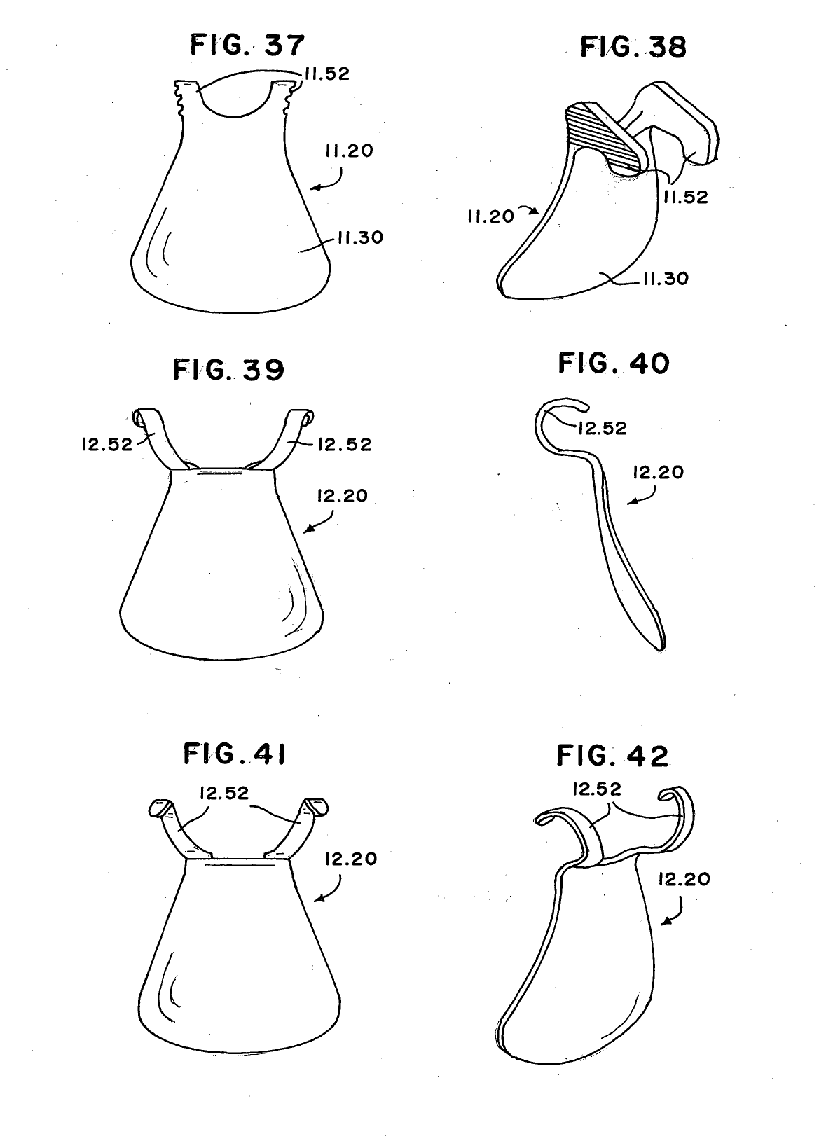

Referring to FIGS. 35-38, an eleventh embodiment 11.20 of the childbirth instrument of the present invention is shown. Eleventh embodiment 11.20 is substantially similar to fourth embodiment 4.20 except that eleventh embodiment 11.20 has no aperture corresponding to aperture 4.54, and a pair of grips 11.52 are provided, being joined to upper portion 11.56 of guard portion 11.30.

Referring to FIGS. 39-42, a twelfth embodiment 12.20 of the childbirth instrument of the present invention is shown. Twelfth embodiment 12.20 is substantially similar to eleventh embodiment 11.20 except that the grips 12.52 of the twelfth embodiment 12.20 are curved rather than straight.

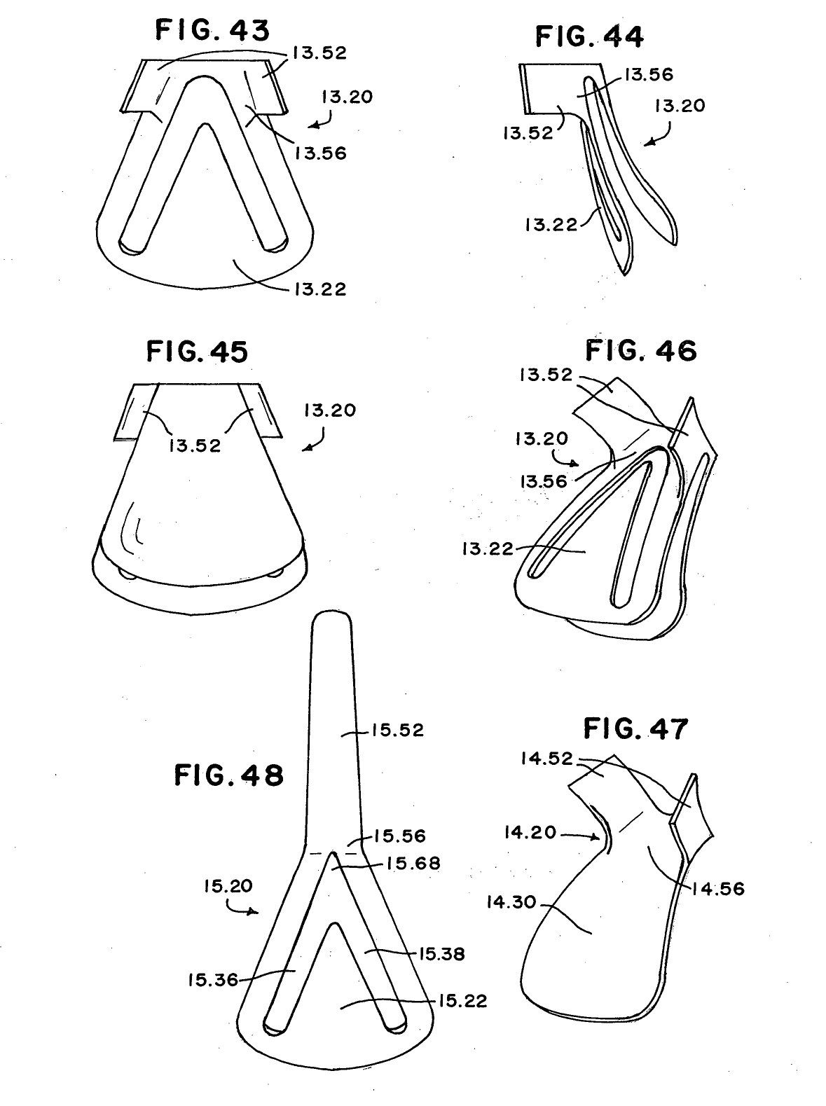

Referring to FIGS. 43-46, a thirteenth embodiment 13.20 of the childbirth instrument of the present invention is shown. Thirteenth embodiment 13.20 is substantially similar to first embodiment 1.20 except that a pair of grips 13.52 are provided, being joined to upper portion 13.56 of guide portion 13.22 and extending remote from guide portion 13.22.

Referring to FIG. 47, a fourteenth embodiment 14.20 of the childbirth instrument of the present invention is shown. Fourteenth embodiment 14.20 is substantially similar to eleventh embodiment 11.20 except that the pair of grips 14.52 that are joined to upper portion 14.56 of guard portion 14.30 extend in an anterior direction, extending remote from guard portion 14.30, rather than in a posterior direction as with embodiment 11.20. As previously noted, the grips of any embodiment may equivalently extend in the anterior or posterior directions, as desired, without departing from the nature and scope of the present invention.

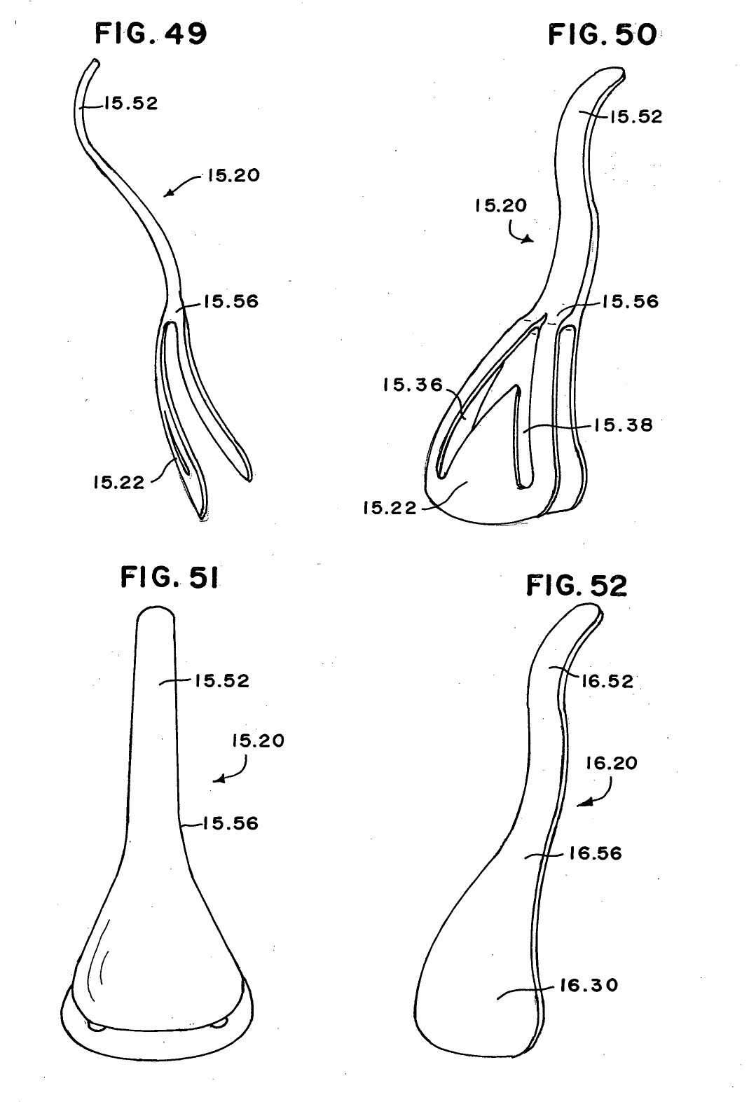

Referring to FIGS. 48-51, a fifteenth embodiment 15.20 of the childbirth instrument of the present invention is shown. Fifteenth embodiment 15.20 is substantially similar to first embodiment 1.20 except that a grip 15.52 is provided, being joined to upper portion 15.56 of guide portion 15.22 and extending remote from guide portion 15.22. As a minor variation, the common upper end 15.68 of first and second elongated reference slots 15.36, 15.38 extend further upward in embodiment 15.20 than in embodiment 1.20, simply to show that the reference slots may be lengthened or shortened as desired to present a desired exposure of the vagina and perineum for the episiotomy incisions through the reference slots, without departing from the nature and scope of the present invention.

Referring to FIG. 52, a sixteenth embodiment 16.20 of the childbirth instrument of the present invention is shown. Sixteenth embodiment 16.20 is substantially similar to fourteenth embodiment 14.20 except that only a single grip 16.52 is provided, being joined to upper portion 16.56 of guard portion 16.30 and extending in a posterior direction remote from guard portion 16.30

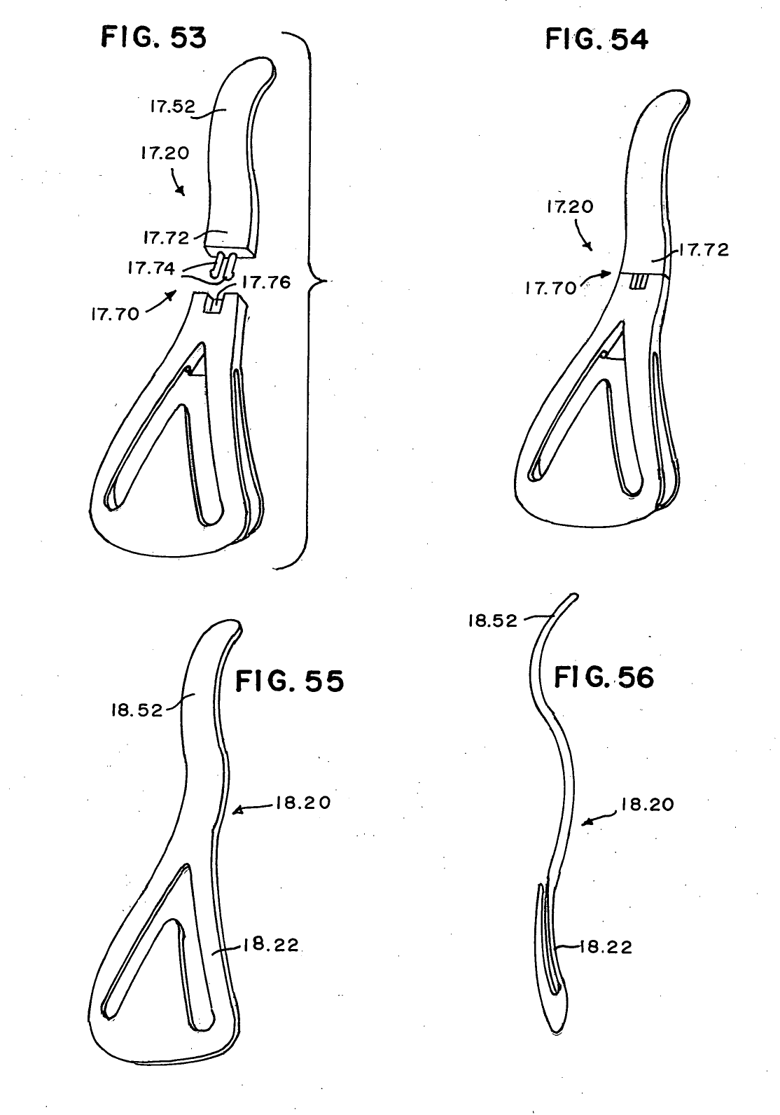

Referring to FIGS. 53-54, a seventeenth embodiment 17.20 of the childbirth instrument of the present invention is shown. Seventeenth embodiment 17.20 is substantially similar to fifteenth embodiment 15.20 except that the seventeenth embodiment 17.20 further includes grip engagement means 17.70 for selectively engaging and disengaging grip 17.52 to and from childbirth instrument 17.20. In the preferred embodiment shown, proximal end 17.72 of grip 17.52 has a pair of outwardly-extending fingers 17.74 that engage and interlock with a mating slot 17.76 in the instrument 17.20.

Referring to FIGS. 55-56, an eighteenth embodiment 18.20 of the childbirth instrument of the present invention is shown. Eighteenth embodiment 18.20 is substantially similar to fifteenth embodiment 15.20 except that there is only a guide portion 18.22 with grip 18.52 extending in a posterior direction remote from guide portion 18.22, and there is no guard portion in the eighteenth embodiment.

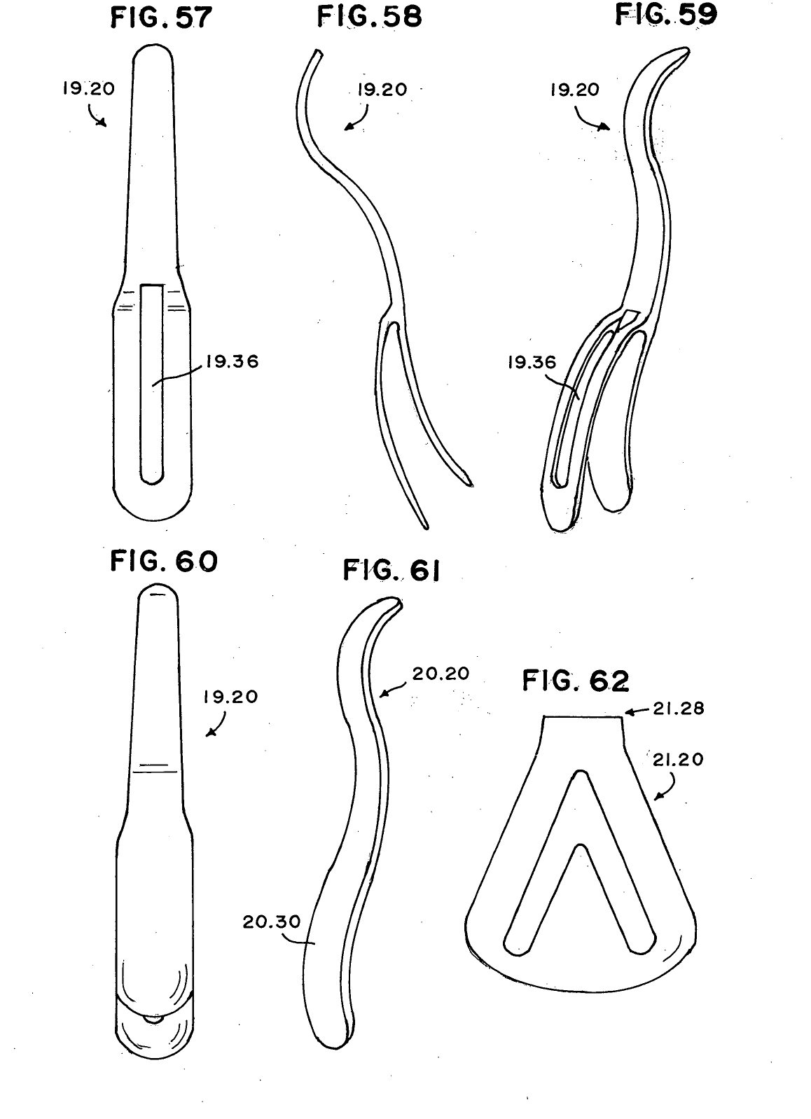

Referring to FIGS. 57-60, a nineteenth embodiment 19.20 of the childbirth instrument of the present invention is shown. Nineteenth embodiment 19.20 is substantially similar to fifteenth embodiment 15.20 except that there is only a single elongated reference slot 19.36 through guide portion 19.22. The nineteenth embodiment 19.20 allows the physician to make the episiotomy incision at an arbitrary angle, and the physician simply docks the instrument 19.20 to the vagina as with the fifteenth embodiment, and uses the reference slot 19.36 to make the episiotomy incision without having a second reference slot in alignment with the anus, thereby providing the guide and guard functions and constraining the length of the episiotomy incision without constraining the angle of the episiotomy incision.

Referring to FIG. 61, a twentieth embodiment 20.20 of the childbirth instrument of the present invention is shown. Twentieth embodiment 20.20 is substantially similar to the sixteenth embodiment 16.20 except that the guard portion 20.30 of embodiment 20.20 is narrower than the guard portion 16.30 of the sixteenth embodiment 16.20.

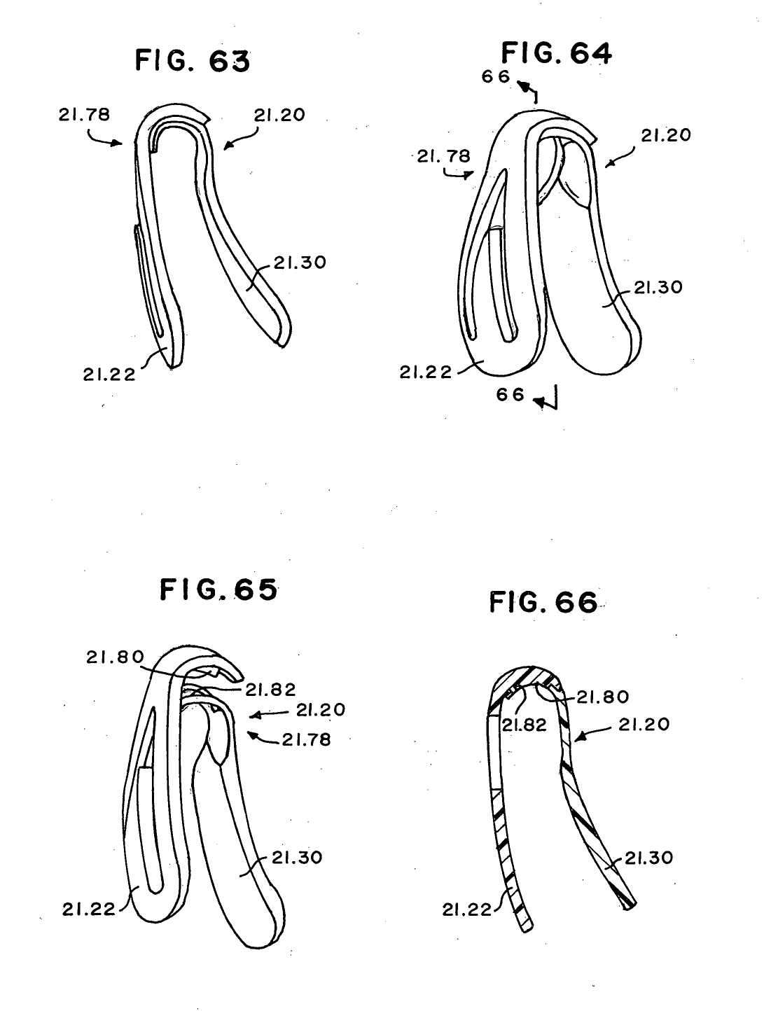

Referring to FIGS. 62-66, a twenty-first embodiment 21.20 of the childbirth instrument of the present invention is shown. Twenty-first embodiment 21.20 is substantially similar to first embodiment 1.20 except that the twenty-first embodiment 21.20 further includes guide-guard engagement means 21.78 for selectively joining and disengaging the guide portion 21.22 to and from guard portion 21.30 at vaginal end 21.28 of instrument 21.20. In the preferred embodiment shown, guide portion 21.30 has a tab 21.80 that engages and interlocks with a mating slot 21.82 in the instrument 21.20.

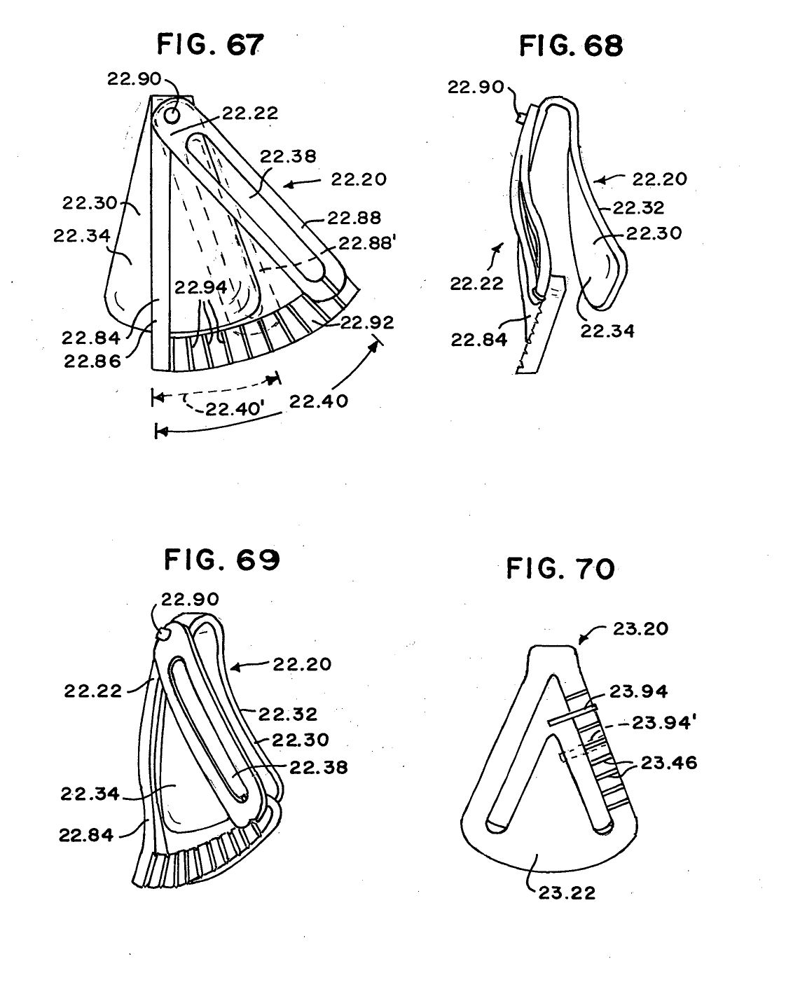

Referring to FIGS. 67-69, a twenty-second embodiment 22.20 of the childbirth instrument of the present invention is shown. Like many of the embodiments, embodiment 22.20 includes a guard portion 22.30 adapted for insertion into a woman's vagina, with guard portion 22.30 being substantially thin and having a concave posterior side 22.32 and having a convex anterior side 22.34. Twenty-second embodiment 22.20 provides for an adjustable angle of the episiotomy incision, and guide portion 22.22 includes a reference member 22.84 joined to guard portion 22.30 at an upper portion 22.56 thereof. Reference member 22.84 has an outer reference edge 22.86, and guide portion 22.22 further includes a guide arm 22.88 pivotally mounted as by a rivet or pin 22.90 to reference member 22.84 for angular moment with respect to reference member 22.84. Guide arm 22.88 has an elongated reference slot 22.38 therethrough, and, as guide arm 22.88 moves about its pivot 22.90, shown in a moved position as 22.88′, reference slot 22.38 is seen to be movable through a plurality of acute angles with respect to reference edge 22.86, moving from an acute angle 22.40 to a plurality of lesser acute angles 22.40′ as shown in dotted outline in FIG. 67. Reference member 22.84 may be provided with a reference scale 22.92 marked with reference angular markings 22.48 so that a desired episiotomy angle 22.40 may be selected. Use of twenty-second embodiment 22.20 is similar to use of the first embodiment 1.20, with reference edge 22.86 being used rather than first reference slot 1.36 for alignment/guiding of the episiotomy cut and with adjustable reference slot 22.38 being used rather than second reference slot 1.38. As before, acute angle 22.40 may be any angle between five degrees and seventy-five degrees, inclusive, but the preferred angle is between fifteen and sixty degrees, inclusive, and the optimal angle is between thirty and forty-five degrees, inclusive.

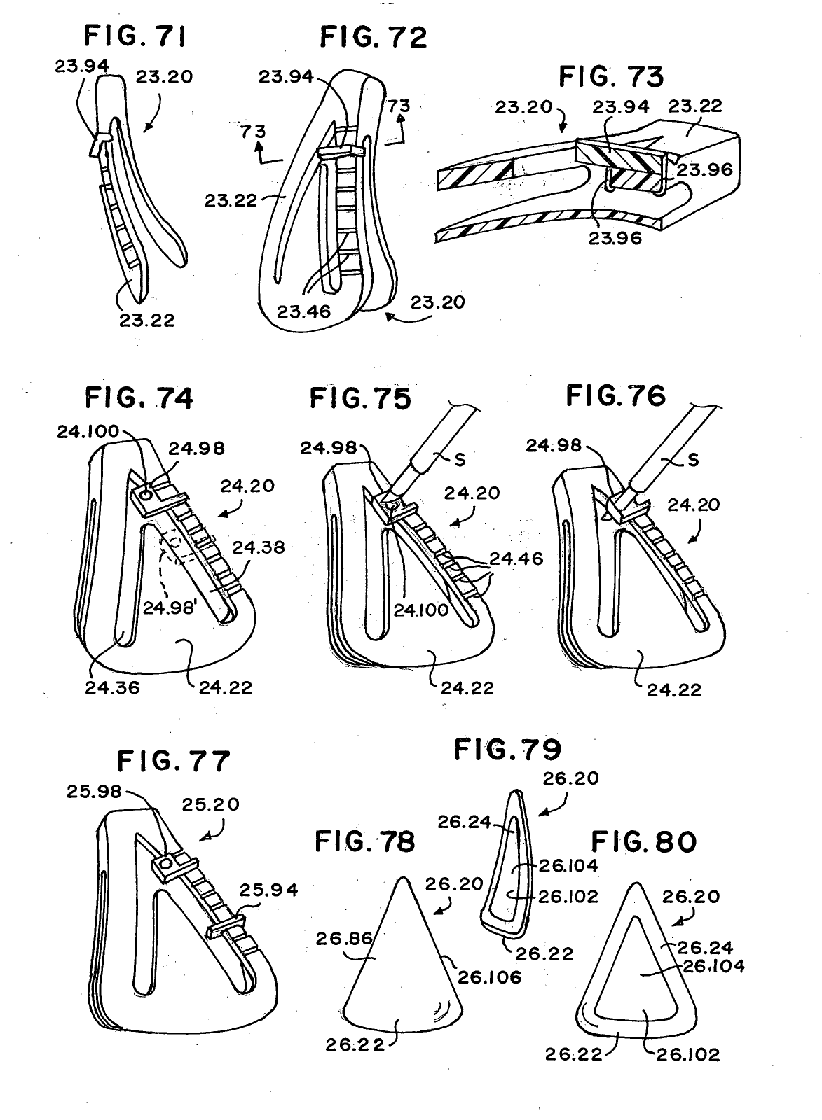

Referring to FIGS. 70-73, a twenty-third embodiment 23.20 of the childbirth instrument of the present invention is shown. Twenty-third embodiment 23.20 is substantially similar to first embodiment 1.20, but the guide portion 23.22 of twenty-third embodiment 23.20 further includes at least one stop 23.94 mounted for sliding movement adjacent and substantially parallel to one or both of elongated reference slots 23.36, 23.38, with the stop being shown in dotted outline as 23.94′ in a moved position in FIG. 70. Stop 23.94 may be mounted to guide portion 23.22 as by a plurality of spaced fingers 23.96 that grip guide portion 23.22 as seen best in FIG. 73, and may be locked into position to prevent over-cutting to an excessive incision length. As with the alternate embodiment 1.20A shown in FIG. 4A, reference linear dimensional markings 23.46 may be provided to allow the position of the stop 23.94 to be set at a desired position, thereby setting the length of the episiotomy incision.

Referring to FIGS. 74-76, a twenty-fourth embodiment 24.20 of the childbirth instrument of the present invention is shown. Twenty-fourth embodiment 24.20 is substantially similar to first embodiment 1.20, but the guide portion 24.22 of twenty-fourth embodiment 24.20 further includes at least one guide member 24.98 mounted for sliding movement adjacent and substantially parallel to one or both of elongated reference slots 24.36, 24.38, with the guide member being shown in dotted outline as 24.98′ in a moved position in FIG. 74. Guide member 24.98 may preferably be mounted to guide portion 24.22 in a manner similar to the mounting of the stop 23.94 as heretofore described for the twenty-third embodiment. As with the alternate embodiment 1.20A shown in FIG. 4A, reference linear dimensional markings 24.46 may be provided to allow the position of the guide member 24.98 to be set at a desired position, thereby setting the length of the episiotomy incision. Guide member 24.98 has a guide aperture 24.100 therethrough adapted for receipt of a surgical instrument such as a scalpel S shown being inserted in FIGS. 75 and 76, and guide member 24.98, with scalpel S thus inserted into guide aperture 24.100, may be moved along the reference slot, thereby permitting a precision incision to be made as the guide member 24.98 is moved with the scalpel S inserted thereinto.

Referring to FIG. 77, a twenty-fifth embodiment 25.20 of the childbirth instrument of the present invention is shown. Twenty-fifth embodiment 25.20 is similar to embodiments 23.20 and 24.20, and shows that both a stop 25.94 and a guide member 25.98 may be included on guide portion 25.22.

Referring to FIGS. 78-80, a twenty-sixth embodiment 26.20 of the childbirth instrument of the present invention is shown. Twenty-sixth embodiment 26.20 has no guard portion and is seen to include a guide portion 26.22 adapted for fitting against a woman's perineal outer surface as best seen in FIG. 82. Guide portion 26.22 is substantially thin and has a concave posterior side 26.24, and posterior side 26.24 has an adhesive 26.102. Adhesive 26.102 may be a sterile removable glue applied to posterior side 26.24 but, in the preferred embodiment, adhesive 26.102 is an adhesive backing 26.104, such as well-known double-faced adhesive tape, applied to the posterior side 26.24 of embodiment 26.20. Guide portion 26.22 has a first reference edge 26.86 and a second reference edge 26.106, and first reference edge is at an acute first angle 26.40 with respect to second reference edge 26.106.

Referring to FIGS. 81-85, the methods of use of various embodiments of the childbirth instrument are shown.

FIGS. 81 and 85 show use of those embodiments of the invention having elongated reference slots, and first embodiment 1.20 will be used as an example of a first embodiment of the method of using the present invention. It shall be understood that the use of all embodiments with elongated reference slots is similar (except for the second embodiment 2.20, whose use is described separately hereinafter), and a description of the use of the first embodiment 1.20 will suffice for all. The difference between FIG. 81 and FIG. 85 is that, in FIG. 81, a reference edge is placed in alignment with the woman's anus when positioning the childbirth instrument, and that, in FIG. 85, a reference slot is placed in alignment with the woman's anus when positioning the childbirth instrument. Both are alternate ways of performing the method of using those embodiments of the invention having elongated reference slots, as hereinafter, explained.

To practice this first embodiment of the method of using the childbirth instrument of the present invention, the childbirth instrument is provided. If the childbirth instrument has a guard, the guard portion is inserted into the woman's vagina as best seen in FIG. 84 so as to protect the unborn infant I from injury. As with all embodiments of the present invention having elongated reference slots, both of the left and right reference slots 1.36, 1.38 have respective reference edges 1.86, 1.106 adjacent thereto. The posterior side of the guide portion is placed in engagement with the woman's perineal outer surface 200 adjacent the woman's vagina V and with a respective reference edge 1.86, 1.106, which is adjacent a chosen one of the first and second reference slots 1.36, 1.38, being in alignment with the woman's anus A, as shown in solid and dotted outline in FIG. 81, depending on whether the episiotomy incision, at the option of the attending physician, is desired to be to the left of the anus or to the right. With the instrument thus positioned, a first episiotomy incision is made to the woman's vagina through the other of the first and second reference slots 1.36, 1.38. If the attending physician/midwife prefers to make two shorter episiotomy incisions rather than, as is usually the case, one longer episiotomy incision, the childbirth instrument is then repositioned so that the other of the respective reference edges 1.86, 1.106, which is adjacent the other of the first and second reference slots 1.36, 1.38, becomes in alignment with the woman's anus A, as shown in dotted outline in FIG. 81 as 1.20′. With the instrument thus repositioned, a second episiotomy cut is-made to the woman's vagina through the chosen one of first and second reference slots 1.36, 1.38. Alternately, as shown in FIG. 85, a chosen one of the first and second reference slots 1.36, 1.38, rather than the respective reference edge, is placed in alignment with the woman's anus A, and the first episiotomy cut is made through the other of the first and second reference slots 1.36, 1.38. As before, if the attending physician/midwife desires to make a second episiotomy cut, the instrument is repositioned as shown in FIG. 85, with the other of the first and second reference slots 1.36, 1.38 being in alignment with the woman's anus, and a second episiotomy cut is made through the chosen one of the first and second reference slots 1.36, 1.38. With the term “reference guide” 1.108 being defined as the group consisting of “(a) a chosen one of the first and second reference slots 1.36, 1.38, and (b) the reference edge adjacent the chosen one of the first and second reference slots 1.36, 1.38”, the method step of positioning of the instrument 1.20 as shown in FIGS. 81 and 85 is understood to be accomplished by having reference guide 1.108 be in alignment with the woman's anus A, where the reference guide 1.108 is selected from the group consisting of (a) a chosen one of the first and second reference slots 1.36, 1.38, and (b) the reference edge adjacent the chosen one of the first and second reference slots 1.36, 1.38. The first episiotomy cut is then performed through the other of the first and second reference slots 1.36, 1.38.

It should be understood that this method permits the left episiotomy incision to be done first and then, after repositioning the instrument, the right episiotomy incision to be done, or, depending on the preference of the attending physician and/or midwife, the right episiotomy incision may be done first, with the instrument positioned as shown in dotted outline as 1.20′, and then the instrument may be repositioned as shown in solid outline as 1.20 in FIG. 81, and then the left episiotomy incision done second.

FIG. 82 shows the second embodiment of the method of using the present invention, specifically, the method of use when the twenty-sixth embodiment 26.20 of the present invention is used.

This second embodiment of the method of using the present invention includes the steps of providing a childbirth instrument of the twenty-sixth embodiment 26.20, then positioning instrument 26.20 upon the woman's perineal outer surface 200 adjacent the woman's vagina V, with adhesive 26.102 being in engagement with the woman's perineal outer surface 200 and with a chosen one of first and second reference edges 26.86, 26.106 being in alignment with the woman's anus A. Then, with the instrument 26.20 thus positioned as shown in solid outline in FIG. 82, the first episiotomy cut is made to the woman's vagina along the other of the first and second reference edges 26.86, 26.106, taking care not to injure the unborn infant I. If desired, a well-known “skin marker” may be used by the attending physician or midwife to mark the angled episiotomy line on the perineum along the other of the first and second reference edges 26.86, 26.106, and the first episiotomy cut can be performed with instrument 26.20 in place or along the marked line after removal of the instrument 26.20.

If a second episiotomy incision is desired, at the option of the attending physician/midwife, the childbirth instrument 26.20 is then repositioned so that the other of the first and second reference edges 26.86, 26.106 becomes in alignment with the woman's anus A and so that the chosen one of the first and second reference edges 26.86, 26.106 is no longer in alignment with the woman's anus A, as shown in dotted outline in FIG. 82 as 26.20′, with adhesive 26.102 being in engagement with the woman's perineal outer surface 200. With the instrument thus repositioned, the second episiotomy cut is made to the woman's vagina along the chosen one of the first and second reference edges 26.86, 26.106, again, taking care not to injure the unborn infant I. Again, as before, if desired, a well-known “skin marker” may be used by the attending physician or midwife to mark the angled episiotomy line on the perineum along the chosen one of the first and second reference edges 26.86, 26.106, and the second episiotomy cut can be performed with instrument 26.20 in place or along the marked line after removal of the instrument 26.20.

It should be understood that this method permits a left episiotomy incision to be done first and then, after repositioning the instrument, a right episiotomy incision to be done, or, depending on the preference of the attending physician and/or midwife, a right episiotomy incision may be done first, with the instrument positioned as shown in dotted outline as 26.20′, and then, if a second episiotomy incision is desired, the instrument may be repositioned as shown in solid outline as 26.20 in FIG. 82, and then a left episiotomy incision done second.

FIG. 83 shows the third embodiment of the method of using the present invention, specifically, the method of use when the second embodiment 2.20 of the present invention is used.

To practice this third embodiment of the method of using the childbirth instrument 2.20 of the present invention, the childbirth instrument 2.20 is first provided and the guard portion 2.30 is inserted into the woman's vagina V to provide protection to the unborn infant I, with the posterior side 2.24 of the guide portion 2.22 being in engagement with the woman's perineal outer surface 200 and with the second reference slot 2.38 being in alignment with the woman's anus A.

With the instrument 2.20 thus positioned, the first episiotomy cut is made to the woman's vagina through a chosen one of the first and third reference slots 2.36, 2.50. If a second episiotomy incision is desired, then, without repositioning childbirth instrument 2.20, a second episiotomy cut is made to the woman's vagina through the other of said first and third reference slots 2.36, 2.50.

It should be understood that this method permits a left episiotomy incision to be done first and then, if a second episiotomy incision is desired, without repositioning the instrument, a right episiotomy incision to be made, or, depending on the preference of the attending physician and/or midwife, a right episiotomy incision may be done first and then, if a second episiotomy incision is desired, a left episiotomy incision done second, without repositioning the instrument 2.20.

The method of using the seventh, eighth, and ninth embodiments 7.20, 8.20, and 9.20 to administer a local anesthetic prior to or during an episiotomy procedure is similar to the use of the first method described hereinbefore. One of embodiments 7.20, 8.20, and 9.20 is provided, and the guard portion is inserted into the woman's vagina to protection to the unborn infant, with the guide portion of the childbirth instrument being outside the woman's vagina. A hypodermic needle of a syringe is then inserted through the aperture 7.66, 8.66, or 9.66, as appropriate, and a local anesthetic is then delivered into the woman's perineum through the hypodermic needle. It should be understood that this method of local anesthesia delivery to the perineum is equally applicable while performing surgical procedures other than episiotomy procedures.

Although the present invention has been described and illustrated with respect to a preferred embodiment and a preferred use therefor, it is not to be so limited since modifications and changes can be made therein which are within the full intended scope of the invention.

We claim:

1. A childbirth instrument comprising a guide portion adapted for fitting against a woman's perineal outer surface, said guide portion having an elongated first reference slot therethrough and an elongated second reference slot therethrough, said first reference slot being at an acute first angle with respect to said second reference slot and said first reference slot being in communication with said second reference slot.

2. The childbirth instrument as recited in claim 1, in which said childbirth instrument further comprises a guard portion, said guard portion being adapted for insertion into a woman's vagina and being joined to said guide portion and spaced apart therefrom, said guard portion and said guide portion being substantially thin and each having a concave posterior side, said guard portion having a convex anterior side opposed from said posterior side of said guide portion.

3. A method of using the childbirth instrument as recited in claim 2, said method comprising the steps of:
(a) providing the childbirth instrument as recited in claim 2, each of said first and second reference slots having a respective reference edge adjacent thereto; then
(b) inserting said guard portion into said woman's vagina with said posterior side of said guide portion being in engagement with said woman's perineal outer surface and with a reference guide being in alignment with said woman's anus, said reference guide being selected from the group consisting of:
i. a chosen one of said first and second reference slots; and
ii. said respective reference edge adjacent said chosen one of said first and second reference slots;
then
(c) performing a first episiotomy cut of said woman's vagina through the other of said first and second reference slots.

4. The childbirth instrument as recited in claim 2, in which said childbirth instrument comprises guide-guard engagement means for selectively joining and disengaging said guide portion to and from said guard portion.

5. The childbirth instrument as recited in claim 2, in which said guide portion includes a stop mounted for sliding movement adjacent and substantially parallel to one of said reference slots.

6. The childbirth instrument as recited in claim 5, in which said guide portion includes a guide member mounted for sliding movement adjacent and substantially parallel to said one of said reference slots, said guide member having a guide aperture therethrough adapted for receipt of a surgical instrument.

7. The childbirth instrument as recited in claim 2, in which said guide portion includes a guide member mounted for sliding movement adjacent and substantially parallel to one of said reference slots, said guide member having a guide aperture therethrough adapted for receipt of a surgical instrument.

8. The childbirth instrument as recited in claim 2, in which said childbirth instrument further has an elongated third reference slot therethrough, said second reference slot being intermediate said first reference slot and said third reference slot, said third reference slot being at said acute first angle with respect to said second reference slot and said third reference slot being in communication with said first and said second reference slots.

9. A method of using the childbirth instrument as recited in claim 8, said method comprising the steps of:
(a) providing the childbirth instrument as recited in claim 8; then
(b) inserting said guard portion into said woman's vagina with said posterior side of said guide portion being in engagement with said woman's perineal outer surface and with said second reference slot in alignment with said woman's anus; then
(c) performing a first episiotomy cut of said woman's vagina through a chosen one of said first and third reference slots.

10. The childbirth instrument as recited in claim 1, in which said childbirth instrument further has reference dimensional markings adjacent at least one of said reference slots.

11. The childbirth instrument as recited in claim 10, in which said childbirth instrument has reference angular markings within said first angle.

12. The childbirth instrument as recited in claim 1, in which said childbirth instrument further comprises a grip extending remote from said guide portion.

13. The childbirth instrument as recited in claim 12, in which said childbirth instrument comprises grip engagement means for selectively engaging and disengaging said grip to and from said childbirth instrument.

14. A childbirth instrument comprising a guard portion adapted for insertion into a woman's vagina, said guard portion being substantially thin and having a concave posterior side and a convex anterior side.

15. The childbirth instrument as recited in claim 14, in which said guard portion further has first and second finger-receiving concave portions on said posterior side, said first and said second finger-receiving portions being spaced apart.

16. The childbirth instrument as recited in claim 15, in which said first and second finger-receiving portions each has a plurality of transverse ribs thereon.

17. The childbirth instrument as recited in claim 16, in which said childbirth instrument further comprises a grip extending from said guard portion.

18. The childbirth instrument as recited in claim 14, in which said childbirth instrument further comprises a grip extending remote from said guard portion.

19. The childbirth instrument as recited in claim 18, in which said childbirth instrument has an aperture therethrough proximate an upper portion of said guard portion and proximate said grip.

20. A method of using the childbirth instrument as recited in claim 19, said method comprising the steps of:
(a) providing the childbirth instrument as recited in claim 19; then
(b) inserting said guard portion into said woman's vagina; then
(c) inserting said hypodermic needle through said aperture; and then
(d) delivering a local anesthetic to said woman's perineum through said hypodermic needle.

21. The childbirth instrument as recited in claim 14, in which said childbirth instrument further comprises a guide portion, said guide portion comprising a reference member joined to said guard portion, said reference member having a reference edge, and said guide portion further comprising a guide arm pivotally mounted to said reference member, said guide arm having an elongated reference slot therethrough, said reference slot being movable to a plurality of acute angles with respect to said reference edge.

22. A childbirth instrument comprising a guide portion adapted for fitting against a woman's perineal outer surface and a guard portion, said guard portion being adapted for insertion into a woman's vagina and being joined to said guide portion and spaced apart therefrom, said guard portion being substantially thin and having a concave posterior side, said guard portion having a convex anterior side opposed from a posterior side of said guide portion, said guide portion having a first aperture therethrough adapted for receipt of a hypodermic needle.

23. A method of using the childbirth instrument as recited in claim 22, said method comprising the steps of:
(a) providing the childbirth instrument as recited in claim 22; then
(b) inserting said guard portion into said woman's vagina with said guide portion being outside said woman's vagina; then
(c) inserting said hypodermic needle through said first aperture; and then
(d) delivering a local anesthetic to said woman's perineum through said hypodermic needle.

24. A childbirth instrument comprising a guide portion adapted for fitting against a woman's perineal outer surface, said guide portion being substantially thin and having a concave posterior side, said posterior side of said guide portion having an adhesive, said guide portion having a first reference edge and a second reference edge, said first reference edge being at an acute first angle with respect to said second reference edge.

25. A method of using the childbirth instrument as recited in claim 24, said method comprising the steps of:
(a) providing the childbirth instrument as recited in claim 24; then
(b) positioning said childbirth instrument upon said woman's perineal outer surface adjacent said woman's vagina with said adhesive being in engagement with said woman's perineal outer surface and with a chosen one of said first and second reference edges being in alignment with said woman's anus; then
(c) performing a first episiotomy cut of said woman's vagina along the other of said first and second reference edges.
